(19) United States
(12) Patent Application Publication
(10) Pub. No.: US 2010/0008959 A1
(43) Pub. Date: Jan. 14, 2010

(54) Cosmetic and Therapeutic Stick Products

(75) Inventors: Ralph Spindler, Palatine, IL (US); Stephen J. Urbanec, Arlington Heights, IL (US); Limin Liu, Palatine, IL (US); Gholam-Reza Vakili-Tahami, Naperville, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: AMCOL INTERNATIONAL CORPORATION, Arlington Heights, IL (US)

(21) Appl. No.: 12/282,535

(22) PCT Filed: Apr. 2, 2007
(86) PCT No.: PCT/US07/08164

§ 371 Date: May 4, 2009

Related U.S. Application Data
(60) Provisional application No. 60/788,925, filed on Apr. 4, 2006.

(51) Int. Cl. A61K 8/02; A61Q 11/00; A61K 33/40

(52) U.S. Cl. 424/401; 424/49; 424/616

(57) ABSTRACT

A composition wherein phase or chemically incompatible components are introduced into a stick composition through the use of a microparticle delivery system.

### CROSS-REFERENCE TO RELATED APPLICATIONS

This application claims the benefit of U.S. Provisional Patent Application No. 60/788,925, filed Apr. 4, 2006.

### FIELD OF THE INVENTION

The present invention is directed to stick compositions comprising invisible or incompatible components through the use of a microparticle delivery system.

### BACKGROUND OF THE INVENTION

A solid stick composition is a convenient way to deliver cosmetics, skin treatments, and pharmaceuticals to the surface of the skin, lips, and teeth. A stick format is easy to store and transport, and does not leak from a container like many emulsion and liquid products. A significant problem in providing stick compositions arises when attempting to incorporate active agents that are immiscible or incompatible with the remainder of the ingredients of the stick composition.

For example, it is very difficult to incorporate a water-soluble active agent into a base stick formulation that is essentially nonpolar, i.e., hydrophobic, in nature. This difficulty arises because the active agent is insoluble in the base stick formulation. If the active agent is a solid, one approach is to mill the solid active agent to a sufficiently fine powder size such that the agent cannot be felt when the stick composition is applied to the skin or lips.

For many solid active agents, it is very difficult, dangerous, and/or costly to mill the active agent to a sufficiently small particle size to overcome the negative esthetic aspects. One example is benzoyl peroxide, which is a hydrophilic compound having a poor solubility in hydrophobic and many hydrophilic materials, but is a very effective active agent in treating acne. Benzoyl peroxide is a shock sensitive material, and, accordingly, milling the compound to a fine particle size is a very difficult and dangerous operation. Therefore, a need exists to incorporate benzoyl peroxide, in an esthetically acceptable form, into a stick composition in a safe manner.

Alternatively, a second approach is to first dissolve a water-soluble (hydrophilic) active agent in a water phase, then incorporate the resulting water phase into a molten waxy (hydrophobic) phase through the use of a water-in-oil (w/o) emulsifier to provide a water-in-oil emulsion. This approach is discussed in U.S. Pat. No. 6,613,338 and U.S. Patent Publication No. 2004/0258721, wherein appropriate emulsifiers to make such stick compositions are described.

However, incorporation of 30% to 85% water, by weight, into a stick composition can be very difficult because many sticks are prepared at elevated temperatures of up to 90° C. to 100° C. For example, if color matching is required, as with pigmented products, the long time that a stick composition is held at an elevated temperature can result in partial or complete evaporation of the water phase. This is a significant technical limitation in the production of stick products on a commercial scale. Furthermore, the above patent documents do not disclose a method for incorporating a hydrophobic active agent into an essentially hydrophilic base stick formulation.

Another approach is described in U.S. Pat. No. 4,678,663, wherein hydrophilic materials are incorporated into a stick composition that includes volatile silicone materials to provide a good skin feel by incorporating sufficient quantities of water-soluble materials, such as polyethylene glycol (molecular weight of 1000), together with an appropriate emulsifier. This approach does not allow the preparation of stick compositions that are free of emulsifiers and hydrophilic phases in addition to the active agent.

### SUMMARY OF THE INVENTION

The present invention is directed to cosmetic, skin treatment, and pharmaceutical stick compositions. More particularly, the present invention is directed to a stick composition containing active agents that are not easily incorporated into a base stick formulation because of a poor solubility, or an incompatibility, of the active agent in the base stick formulation.

In many cases, a base stick formulation is prepared from a mixture of oils and waxes whose proportion and identity can help determine the skin feel and physical attributes of the final stick product. Examples of this type of formulation are provided in Chapter 22 of “The Chemistry and Manufacture of Cosmetics”, Volume II, Ed. Mitchell Schlossman (2002), wherein a variety of lipstick formulations are discussed. In this case, the base stick composition is considered predominately hydrophobic in nature.

A stick base that is predominately hydrophilic in nature can be made by adding appropriate gelling agents, typically surfactants or soaps, to a mixture of water and polyhydroxy materials, such as glycols. Examples of such stick formulations can be found in U.S. Pat. No. 5,681,552 and U.S. Pat. No. 5,776,475.

In accordance with the present invention, active agents first are loaded onto a microparticle delivery system, then incorporated into a molten base stick formulation. By using this method, hydrophilic active agents can be incorporated into a hydrophobic base stick formulation, and conversely, hydrophobic active agents can be incorporated into a hydrophilic base stick formulation. Additional compounds that are not sufficiently soluble in either hydrophobic or hydrophilic base stick formulations can be loaded first into the microparticle delivery system. In addition, the base stick formulation can comprise a mixture of hydrophobic and hydrophilic components.

It has been found that a microparticle delivery system provides benefits in liquid cosmetic, skin treatment, and pharmaceutical stick compositions. These benefits include improved stability of the active agent, sustained delivery of the active agent to the surface of the skin, lips, or teeth, and an ability to deliver the active agent while simultaneously helping control excess skin oil or adsorption of moisture. Another benefit of incorporating a microparticle delivery system, either free or loaded with an active agent, in a stick formation is that the microparticle delivery system makes the stick product more esthetically appealing because the product feels less greasy on the skin.

These and other novel aspects of the present invention will become apparent from the following detailed description of the preferred embodiments.

### DETAILED DISCUSSION OF THE PREFERRED EMBODIMENTS

Delivery systems routinely are used in cosmetic, skin treatment, and pharmaceutical compositions to extend the useful life of an active agent, to protect the active agent from decomposition in the composition, or to enable or facilitate formulation of the active agent into a composition due to formulation problems, such as solubility or esthetics.

The present invention helps overcome these problems by incorporating a high percentage of an active agent into a polymeric microparticle delivery system, then including the loaded microparticles in a base stick formulation. An active agent is incorporated, i.e., loaded, onto the polymeric microparticles by spraying or adding the active agent directly to the microparticles in a manner such that an essentially homogeneous distribution of the active agent is achieved on the microparticles.

As used herein, the term “base stick formulation” means a stick composition containing all desired ingredients except the microparticle delivery system. The microparticle delivery system can be either unloaded or loaded with one or more compound, e.g., an active agent and/or adjuvant.

In addition, as used herein, the terms “a hydrophilic formulation” or “a hydrophilic agent” mean the compound or agent has a water solubility of at least 0.1 grams per 100 grams of water at 25° C. The terms “a hydrophobic formulation” or “a hydrophobic agent” mean the compound or agent has a water solubility of less than 0.1 grams per 100 grams of water at 25° C.

If the active agent is a solid, the active agent can be dissolved in a suitable volatile solvent. The resulting solution is added to the microparticles, then the volatile solvent is removed, for example, under vacuum with gentle heating. In some cases, this loading process is repeated several times to achieve the desired loading level of the active agent on the microparticles. Another method of loading of a solid active agent that is insufficiently soluble in an appropriate volatile solvent is to disperse the solid active agent in a suitable carrier, such as a polyethylene glycol, then add the dispersion directly to the microparticle delivery system.

A delivery system that can overcome prior problems in the art is based on adsorbent microparticles. Absorbent polymeric microparticles useful in the present invention have an ability to absorb several times their weight of a solid or liquid compound, such as an active agent.

One preferred class of adsorbent polymeric microparticles useful as a delivery system is prepared by a suspension polymerization technique, as set forth in U.S. Pat. Nos. 5,677,407; 5,712,358; 5,777,054; 5,830,967; and 5,834,577, each incorporated herein by reference. Such an adsorbent polymer is sold under the tradename of POLY-PORE® E200 (INCI name: allyl methacrylate crosspolymer) available from AMCOL International Corporation, Arlington Heights, Ill.

Another preferred class of adsorbent microparticle polymers useful as a delivery system is prepared by a precipitation polymerization technique, as set forth in U.S. Pat. Nos. 5,830,960; 5,837,790; 6,248,849; and 6,387,995, each incorporated herein by reference. Such an adsorbent polymer is sold under the tradename POLYTRAP® 7603 (INCI name: glycol dimethacrylate/alkyl methacrylate crosspolymer), also available from AMCOL International Corp.

These adsorbent microparticle polymers also can be modified after the incorporation of an active agent to modify the rate of release of such a compound as set forth in U.S. Pat. No. 6,491,953, incorporated herein by reference.

Another useful class of adsorbent polymers prepared by a precipitation polymerization technique is disclosed in U.S. Pat. Nos. 4,962,170; 4,948,818; and 4,962,133, each incorporated herein by reference, and are commercially available under the tradename of POLYTRAP® 6603 (INCI name: lauryl methacrylate/glycol dimethacrylate crosspolymer), also available from AMCOL International Corp. Other useful, commercially available adsorbent polymers include, for example, MICROSPONGE® (INCI name: methyl methacrylate/glycol dimethacrylate crosspolymer), available from AMCOL International Corp., and Poly-HIPE polymers (e.g., a copolymer of 2-ethylhexyl acrylate, styrene, and divinylbenzene) available from Biopore Corporation, Mountain View, Calif.

In particular, the adsorbent polymer microparticles prepared by the suspension polymerization technique, e.g., POLY-PORE® E200, are a highly porous and highly crosslinked polymer in the form of open (i.e., broken) spheres and sphere sections characterized by a mean unit particle size of about 0.5 to about 3,000 microns, preferably about 0.5 to about 300 microns, more preferably about 0.5 to about 100 microns, and most preferably about 0.5 to about 80 microns. A significant portion of the spheres is about 20 microns in diameter.

The polymeric microparticles are oil and water adsorbent, and have an extremely low bulk density of about 0.008 gm/cc to about 0.1 gm/cc, preferably about 0.009 gm/cc to about 0.07 gm/cc, and more preferably about 0.0095 gm/cc to about 0.04-0.05 gm/cc. The microparticles are capable of holding and releasing oil soluble or dispersible active agents, as well as water soluble or dispersible active agents, individually, or both oil soluble and water soluble compounds simultaneously.

The adsorbent polymer microparticles prepared by the suspension polymerization technique include at least two polyunsaturated monomers, preferably allyl methacrylate and an ethylene glycol dimethacrylate, and, optionally, monounsaturated monomers. The microparticles are characterized by being open to their interior, due either to particle fracture upon removal of a porogen after polymerization or to subsequent milling. The microparticles have a mean unit diameter of less than about 50 microns, preferably less than about 25 microns, and have a total adsorption capacity for organic liquids, e.g., mineral oil, that is at least about 72% by weight, preferably at least about 93% by weight, and an adsorption capacity for hydrophilic compounds and aqueous solutions of about 70% to about 89% by weight, preferably about 75% to about 89% by weight, calculated as weight of material adsorbed divided by total weight of material adsorbed plus dry weight of polymer. In a preferred embodiment, the broken sphere microparticles are characterized by a mean unit diameter of about I to about 50 microns, more preferably of about 1 to about 25 microns, most preferably, of about 1 to about 20 microns.

Preferred polymeric microparticle delivery systems comprise a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, a copolymer of methyl methacrylate and glycol methacrylate, and mixtures thereof.

Specific polymeric microparticles useful in the present invention can be the previously described POLY-PORE® E200, POLYTRAP® 7603, POLYTRAP® 6603, MICROSPONGE®, or Poly-HIPE particles, for example. An active agent is loaded onto such microparticles to provide microparticles containing about 10% to about 90%, preferably about 20% to about 80%, by weight, of the active agent. The active agent-loaded microparticles typically are incorporated into a stick composition in an amount to provide about 0.05% to about 10%, by weight, of an active agent in the composition.

To provide a delivery system for an active agent, the active agent is incorporated, or loaded, onto or into the microparticles. As used herein, the term “loaded microparticle” refers to a microparticle having an active agent added thereto. Loading of the active ingredient includes one or more of impregnating, imbedding, entrapping, absorbing, and adsorbing of the active ingredient into or onto the polymeric microparticles. Loading of the active agent also can be referred to as an “entrapment.” The term entrapment refers to a physical loading of the active ingredient onto the microparticles. Loading can be accomplished by spraying or adding the active agent directly to the microparticles in a manner such that a homogeneous distribution of the active agent on the microparticles is achieved.

Alternatively, the active agent first can be dissolved in a suitable solvent, then the resulting solution is sprayed or added to the microparticles. The solvent then is removed by heating, vacuum, or both. As previously stated, two or more different types of materials can be added to the microparticles, wherein one of the materials is an active agent and the other material is used either to modify the release rate of the active agent from the microparticles, and/or to protect the active agent loaded in the microparticles from reacting or otherwise interacting with other ingredients contained in the final stick formulation. These release modifying or protective materials can be added directly to the microparticles in their molten state, or first dissolved in a suitable solvent, sprayed onto the microparticles followed by removal of the solvent from the delivery system. Alternatively, an active agent can be mixed with a barrier layer material, then loaded on a microparticle delivery system.

After loading an active agent on the microparticles, a barrier layer, optionally, can be applied to the loaded microparticles to prevent rapid diffusion of active agent from the microparticles, and to protect the active agent from the surrounding environment until application. This is especially effective for reactive active agents.

Examples of materials that can be used as a barrier layer, also termed a secondary loading or secondary entrapment, include, but are not limited to, low melting alcohols (C₈ through C₂₀) and fatty alcohols ethoxylated with one to three moles of ethylene oxide. Examples of fatty alcohols and alkoxylated fatty alcohols include, but are not limited to, biphenyl alcohol, caprylic alcohol, cetyl alcohol, cetaryl alcohol, decyl alcohol, lauryl alcohol, isocetyl alcohol, myristyl alcohol, oleyl alcohol, stearyl alcohol, tallow alcohol, steareth-2, ceteth-1, cetearth-3, and laureth-2. Additional fatty alcohols and alkoxylated alcohols are listed in the International Cosmetic Ingredient Dictionary and Handbook, Tenth Edition, Volume 3, pages 2127 and pages 2067-2073 (2004), incorporated herein by reference.

Another class of materials that can be used a barrier layer is the C₈ to C₁₂ fatty acids, including, but not limited to, stearic acid, capric acid, behenic acid, caprylic acid, lauric acid, myristic acid, tallow acid, oleic acid, palmitic acid, isostearic acid and additional fatty acids listed in the International Cosmetic Ingredient Dictionary and Handbook, Tenth Edition, Volume 3, page 2126-2127 (2004), incorporated herein by reference. The barrier material also can be a hydrocarbon, like mineral oil, 1-decene dimer, polydecene, paraffin, petrolatum, vegetable-derived petrolatum or isoparafin. Another class of barrier materials is waxes, like mink wax, carnauba wax, and candelilla wax, for example, and synthetic waxes, like silicone waxes, polyethylene, and polypropylene, for example.

Fats and oils can be useful barrier material agents, which include, for example, but are not limited to, lanolin oil, linseed oil, coconut oil, olive oil, menhaden oil, castor oil, soybean oil, tall oil, rapeseed oil, palm oil, and neatsfoot oil, and additional fats and oils listed in the International Cosmetic Ingredient Dictionary and Handbook, Tenth Edition, Volume 3, pages 2124-2126 (2004). Other useful classes of barrier materials include a water-insoluble ester having at least 10 carbon atoms, and preferable 10 to about 32 carbon atoms. Numerous esters are listed in International Cosmetic Ingredient Dictionary and Handbook, Tenth Edition, Volume 3, pages 2115-2123 (2004).

The barrier layer can be about 1 to about 75 wt. %, by total weight of the loaded polymeric microparticles. In a preferred embodiment, the barrier layer is present at about 5 to about 67 wt. %, and more preferably about 10 to about 50 wt. %, by total weight of the loaded polymeric microparticles.

A stick composition of the present invention therefore comprises polymeric microparticles loaded with an active agent and an optional barrier material. The stick composition also can contain other ingredients well known in the art of stick formulation. As stated above, the term “base stick formulation” means all stick composition ingredients other than the loaded or unloaded microparticles.

A wide variety of active agents can be loaded onto polymeric microparticles, and thereby included in a stick composition of the present invention. Examples of active agents include, but are not limited to anti-acne agents, such as salicylic acid, benzoyl peroxide, sulfur, retinoic acid, and resorcinol; tooth-whitening agents, such as hydrogen peroxide and carbamate peroxide; and skin-treatment agents, such as retinol, retinol palmitate, retinol acetate, dimethicone, petrolatum, hydroquinone, arbutin, ascorbic acid and derivatives thereof, tocopherol and derivatives thereof, skin lightening agents, such as hydroquinone, arbutin, kojic acid, and kojic dipalmitate, skin darkening agents, such as dihydroxyacetone, and L-erytlrulose. These active agents are incorporated into the stick composition after loading onto the microparticle delivery system.

More particularly, the active agent can be one of, or a mixture of, a cosmetic compound, a medicinally active compound, or any other compound that is useful upon topical application to the skin, lips, or teeth. Such active agents include, but are not limited to, deodorants, skin-care compounds, antioxidants, antibacterial compounds, antifungal compounds, anti-inflammatory compounds, topical anesthetics, sunscreens, and other cosmetic and medicinal topically effective compounds.

The active agent can be a deodorant or antiperspirant compound, such as an astringent salt or a bioactive compound. The astringent salts include organic and inorganic salts of aluminum, zirconium, zinc, and mixtures thereof. The anion of the astringent salt can be, for example, sulfate, chloride, chlorohydroxide, alum, formate, lactate, benzyl sulfonate, or phenyl sulfonate. Exemplary classes of antiperspirant astringent salts include aluminum halides, aluminum hydroxyhalides, zirconyl oxyhalides, zirconyl hydroxyhalides, and mixtures thereof.

Exemplary aluminum salts include aluminum chloride and the aluminum hydroxyhalides having the general formula Al₂(OH)ₓQ_{y}.XH₂O, wherein Q is chlorine, bromine, or iodine; x is about 2 to about 5; x+y is about 6, wherein x and y are not necessarily integers; and X is about 1 to about 6. Exemplary zirconium compounds include zirconium oxy salts and zirconium hydroxy salts also referred to as zirconyl salts and zirconyl hydroxy salts, and represented by the general empirical formula ZrO(OH)_{2-nz}L_{z}, wherein z varies from about 0.9 to about 2 and is not necessarily an integer; n is the valence of L; 2−nz is greater than or equal to 0; and L is selected from the group consisting of halides, nitrate, sulfamate, sulfate, and mixtures thereof.

Exemplary deodorant compounds, therefore, include, but are not limited to, aluminum bromohydrate, potassium alum, sodium aluminum chlorohydroxy lactate, aluminum sulfate, aluminum chlorohydrate, aluminum-zirconium tetrachlorohydrate, an aluminum-zirconium polychlorohydrate complexed with glycine, aluminum-zirconium trichlorohydrate, aluminum-zirconium octachlorohydrate, aluminum sesquichlorohydrate, aluminum sesquichlorohydrex PG, aluminum chlorohydrex PEG, aluminum zirconium octa-chloro-hydrex glycine complex, aluminum zirconium penta-chlorohydrex glycine complex, aluminum zircon-nium tetrachlorohydrex glycine complex, aluminum zirconium trichlorohydrex glycine complex, aluminum chlorohydrex PG, zirconium chlorohydrate, aluminum dichlorohydrate, aluminum dichlorohydrex PEG, alumi-num dichlorohydrex PG, aluminum sesquichloro-hydrex PG, aluminum chloride, aluminum zirconium penta-chlorohydrate, chlorophyllin copper complex, numerous other useful antiperspirant compounds known in the art, and mixtures thereof

In addition, other compounds can be included as the active agent in an amount sufficient to perform their intended function. For example, if the composition is intended to be a sunscreen, then agents such as benzophenone-3, trihydroxycinnamic acid and salts, tannic acid, uric acids, quinine salts, dihydroxy naphtholic acid, an anthranilate, diethanolamine methoxy-cinnamate, p-aminobenzoic acid, phenylbenzimidazole sulfonic acid, PEG-25, p-aminobenzoic acid, or triethanol-amine salicylate can be used as the active agent.

Further, sunscreen compounds such as oxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petro-latum, titanium dioxide, 4-menthyl-benzylidene camphor, benzophenone-1, benzophenone-2, benzophenone-6, benzophenone-12, isopropyl dibenzoyl methane, butyl methoxydibenzoylmethane, zotocrylene, or zinc oxide can be used as the active agent.

Similarly, topically active drugs, like antifungal compounds, antibacterial compounds, antiinflammatory compounds, topical anesthetics, skin rash, skin disease, and dermatitis medications, and anti-itch and irritation-reducing compounds can be used as the active agent in the stick compositions of the present invention. For example, analgesics such as benzocaine, dyclonine hydrochloride, aloe vera, and the like; anesthetics such as butamben picrate, lidocaine hydrochloride, xylocaine, and the like; antibacterials and antiseptics, such as povidoneiodine, polymyxin b sulfate-bacitracin, zinc-neomycin sulfate-hydrocortisone, chloramphenicol, ethylbenzethonium chloride, erythromycin, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; anti-inflammatory agents, such as alclometasone dipropionate, beta-methasone valerate, and the like; burn relief ointments, such as o-amino-p-toluene-sulfonamide monoacetate, and the like; depigmenting agents, such as monobenzone; dermatitis relief agents, such as the active steroid amcinonide, diflorasone diactate, hydrocortisone, and the like; diaper rash relief agents, such as methylbenzethonium chloride, and the like; emollients and moisturizers, such as mineral oil, PEG-4 dilaurate, lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclormetasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-1-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin bleaching agents, skin protestant, such as allantoin, and anti-acne agents, such as salicylic acid, also can be incorporated in a composition of the present invention in an amount sufficient to perform its intended function. Other topically active compounds are listed in Remington's Pharmaceutical Sciences, 17th Ed., Merck Publishing Co., Easton, Pa. (1985), pages 773-791 and pages 1054-1058 (hereinafter Remington's), incorporated herein by reference.

The stick compositions of the present invention can be in the form of a solid or gel. The gel has sufficient structural integrity to maintain its shape for at least 2 hours, upon standing at 25° C.

### Example 1

Loading of hydrogen peroxide onto polymeric microparticles was performed as follows. A commercially-available 50%, by weight, hydrogen peroxide solution was loaded onto POLY-PORE® E200 polymeric microparticles by direct addition of the hydrogen peroxide solution onto the microparticles. This loading resulted in free-flowing, slightly moist microparticles that contained 37.5%, by weight, hydrogen peroxide. This product is known as POLY-PORE® 337HP.

### Example 2

The following stick composition was prepared by first melting the waxes and oils listed below, i.e., the hydrophobic base stick formulation, to form a molten mixture, then the loaded microparticles of Example 1 were incorporated into the homogeneous molten mixture. The resulting molten composition was poured into an appropriate stick mold to provide a stick composition containing 6%, by weight, hydrogen peroxide.

| Material | Composition (Wt. %) |
|---|---|
| Petrolatum | 45% |
| Stearyl Alcohol | 15% |
| Carnauba Wax | 8% |
| Mineral Oil | 16% |
| POLY-PORE ® 337HP | 16% |

### Example 3

Another hydrophobic stick composition-was prepared in a similar manner as Example 2 to provide the following composition containing 6%, by weight, hydrogen peroxide.

| Material | Composition (Wt. %) |
|---|---|
| Petrolatum | 40% |
| Stearyl Alcohol | 18% |
| Carnauba Wax | 10% |
| Mineral Oil | 16% |
| POLY-PORE ® 337HP | 16% |

### Example 4

A hydrophilic stick composition was prepared in a similar manner as Example 2 to provide the following composition containing 6%, by weight, hydrogen peroxide:

| Material | Composition (Wt. %) |
|---|---|
| PEG-32 | 53.2% |
| Stearyl Alcohol | 24.7% |
| Polaxamer 407 | 4.1% |
| Flavor | 2% |
| POLY-PORE ® 337HP | 16% |

### Example 5

Another hydrophilic stick composition was prepared in a similar manner as Example 2 to provide the following composition containing 6%, by weight, hydrogen peroxide:

| Material | Composition (Wt. %) |
|---|---|
| PEG-32 | 68% |
| Polaxamer 407 | 16% |
| POLY-PORE ® 337HP | 16% |

### Example 6

A skin-lightening stick composition was prepared by incorporating a loading of 40%, by weight, hydroquinone on POLY-PORE® E200 (i.e., POLY-PORE® 340HQ) on the following waxes and oil of a base hydrophobic stick formulation. The composition contained 2%, by weight, hydroquinone.

| Material | Composition (Wt. %) |
|---|---|
| Stearyl Alcohol | 22.5% |
| Petrolatum | 50% |
| Mineral Oil | 22.5% |
| POLY-PORE ® 340HQ | 5% |

### Example 7

A skin-lightening stick was prepared by incorporating a loading of 40%, by weight, hydroquinone in POLY-PORE® E200 (i.e., POLY-PORE® 340HQ) and a loading of 80%, by weight, cyclomethicone in POLY-PORE® E200 (i.e., POLY-PORE® 180CM) in the following hydrophobic base stick formulation. The stick composition contained 2%, by weight, hydroquinone.

| Material | Composition (Wt. %) |
|---|---|
| Stearyl Dimethicone | 75% |
| C30-45 Alkyl Methicone | 50% |
| POLY-PORE ® 180CM | 5% |
| POLY-PORE ® 340HQ | 5% |

### Example 8

A skin treatment or skin-lightening stick composition was prepared by incorporating a loading of 15%, by weight, ascorbic acid in POLY-PORE® E200 (i.e., POLY-PORE® 315AC) and a loading of 80%, by weight, cyclomethicone in POLY-PORE® E200 (i.e., POLY-PORE® 180CM) in the following hydrophobic base stick composition. The stick composition contained 1%, by weight, ascorbic acid.

| Material | Composition (Wt. %) |
|---|---|
| Stearyl Dimethicone | 70.3% |
| C30-45 Alkyl Methicone | 15% |
| POLY-PORE ® 315AC | 6.7% |
| POLY-PORE ® 180CM | 8% |

### Example 9

A skin treatment or skin-lightening stick composition was prepared by incorporating a loading of 15%, by weight, ascorbic acid in POLY-PORE® E200 (i.e., POLY-PORE® 315AC) and a loading of 80%, by weight, cyclomethicone in POLY-PORE® (i.e., POLY-PORE® 180CM) in the following hydrophobic base stick composition. The stick composition contained 2%, by weight, ascorbic acid.

| Material | Composition (Wt. %) |
|---|---|
| Stearyl Dimethicone | 67.3% |
| C30-45 Alkyl Methicone | 14.4% |
| POLY-PORE ® 315AC | 13.3% |
| POLY-PORE ® 180CM | 5% |

### Example 10

A self-tanning stick composition was prepared by incorporating a loading of 50%, by weight, dihydroxyacetone in POLY-PORE® E200 (i.e., POLY-PORE® 350DA) in the hydrophobic following base stick formulation. The composition contained 5%, by weight, dihydroxyacetone.

| Material | Composition (Wt. %) |
|---|---|
| Stearyl Dimethicone | 74.2% |
| C30-45 Alkyl Methicone | 15.8% |
| POLY-PORE ® 350DA | 10% |

### Example 11

A skin-treatment stick composition was prepared by incorporating a loading of 20%, by weight, retinol in POLY-PORE® E200 (i.e., POLY-PORE® 120RE) and a loading of 50%, by weight, salicylic acid in POLY-PORE® E200 (i.e., POLY-PORE® 150SA) in the following hydrophobic base stick formulation. The composition contained 0.1%, by weight, retinol and 2%, by weight, salicylic acid.

| Material | Composition (Wt. %) |
|---|---|
| Stearyl Dimethicone | 77.9% |
| C30-45 Alkyl Methicone | 16.6% |
| POLY-PORE ® 150SA | 4% |
| POLY-PORE ® 120RE | 0.5% |

### Example 12

A anti-acne stick composition was prepared by incorporating a loading of 38%, by weight, benzoyl peroxide in POLY-PORE® E200 (i.e., POLY-PORE® 338HP) and a loading of 80%, by weight, cyclomethicone in POLY-PORE® E200 (i.e., POLY-PORE® 180CM) in the following hydrophobic base stick formulation. The composition contained 2%, by weight, benzoyl peroxide.

| Material | Composition (Wt. %) |
|---|---|
| Stearyl Dimethicone | 74.2% |
| C30-45 Alkyl Methicone | 15.8% |
| POLY-PORE ® 3337HP | 6.7% |
| POLY-PORE ® 180CM | 3.4% |

### Example 13

A clinical trial was performed to determine the effectiveness of the tooth whitening stick composition of Example 4. The lingual surface of human, permanent tooth specimens were flattened with their roots removed. The specimens then were mounted to a Plexiglas rod (¼″ diameter×2″ long) such that the buccal enamel surface was exposed. The specimens then were gently buffed with a 3:2 flour of pumice water slurry to remove exogenous stains. The specimens then were rinsed, placed in plastic containers under humid conditions, and refrigerated until use. The baseline color of each specimen was determined photometrically using a Minolta CM-2600d spectrophotometer. The area examined was the center of the specimen. Four readings per specimen were obtained by turning the specimen 90° for each reading. All values (L*, a*, and b*) were recorded, and the average L* value and ΔE were calculated. The stick composition was applied for six treatments for one hour intervals at 37° C. Two coats of the stick composition were applied to ensure complete coverage. The negative control group was immersed in fresh, pooled saliva during the treatment interval. Following treatment, the specimens were gently brushed with a soft toothbrush and thoroughly rinsed with deionized (DI) water. The specimens then were immersed in pooled human saliva (37° C.) for one hour between treatments. Three treatments were performed per day for a total of two days of treatment. Overnight, the specimens were maintained in a humid environment under refrigerated conditions. Specimens were placed in saliva for 30 minutes before each daily treatment. The data is summarized in the following table.

Data Set

| Group | | Δ L | Δ E |
|---|---|---|---|
| Control | Mean | −1.81 | 3.16 |
| | SD | 1.17 | 1.39 |
| | SEM | 0.34 | 0.40 |
| Example 4 | Mean | 1.88 | 5.05 |
| | SD | 1.72 | 2.20 |
| | SEM | 0.50 | 0.64 |

These results show that the stick composition of Example 4 significantly improved the brightness of the teeth (ΔL) and the overall color of the teeth (ΔE).

### Example 14

To determine the rate of development of a tan on skin, an in vitro technique described by R. Jermann et al., International Journal of Cosmetic Chemistry (2002), 24, pages 1-8, was used. In this method, VITRO-SKIN™ (IMS, Milford, Conn.) was used as a substrate because it is similar to human skin and reacts with dihydroxyacetone (DHA) to form a brown color. Color development was monitored as a function of time by using a color meter. The color meter measured the L*, a*, and b* color parameters which were compared to the same values for the original VITRO-SKIN™ substrate using the following equation: ΔE=((L*(0)−L*(t))2+(a*(0)−a*(t))2+(b*(0)−b*(t))2)½, wherein L*(0) is the brightness value at time 0 before the self-tanning composition has been applied to the substrate and L*(t) is the brightness value at a time (t) after application of the composition, and similar values for a* and b* as a function of time. The rate of tanning, as gauged by ΔE, also changed as a function of time when a 30 mg self-tanning stick of Example 10 composition was applied to the 8.4 cm² of VITRO-SKIN, as shown in the data set in the table below. This data shows that DHA-can be delivered effectively from a stick composition using a microparticle delivery system.

| Time (Hrs) | Delta E |
|---|---|
| 0 | 1.6 |
| 2 | 4.6 |
| 4 | 10.4 |
| 7 | 17.9 |
| 8 | 18.9 |
| 24 | 29.6 |

### Example 15

A anti-acne stick composition was prepared by incorporating a loading of 38%, by weight, benzoyl peroxide in POLY-PORE® E100 with propylene glycol and adding POLYTRAP® 6603 in the following stearyl dimethicone and C30-45 alkyl methicone base stick formulation. The composition contained 2.5%, by weight, benzoyl peroxide.

| Material | Composition (Wt. %) |
|---|---|
| Stearyl Dimethicone | 74.1% |
| C30-45 Alkyl Methicone | 17% |
| POLY-PORE ® 38% Benzoyl Peroxide | 6.5% |
| POLYTRAP 6603 | 2.4% |

Obviously, many modifications and variations of the invention as hereinbefore set forth can be made without departing from the spirit and scope thereof and, therefore, only such limitations should be imposed as are indicated by the appended claims.

What is claimed is:

1. A stick composition comprising a microparticle delivery system and a base stick formulation.

2. The stick composition of claim 1 wherein the microparticle delivery system comprises polymeric microparticles.

3. The stick composition of claim 2 wherein the polymeric microparticles are free of an added active agent.

4. The stick composition of claim 2 wherein the polymeric microparticles are loaded with an added active agent.

5. The stick composition of claim 4 wherein the base stick formulation is hydrophobic and the active agent is primarily hydrophilic.

6. The stick composition of claim 4 wherein the base stick formulation is hydrophilic and the active agent is primarily hydrophobic.

7. The stick composition of claim 4 wherein the base stick formulation is hydrophobic or hydrophilic and the active agent is insoluble in the base stick formulation.

8. The stick composition of claim 2 wherein the polymeric microparticles are highly crosslinked and are derived from methacrylate monomers, acrylate monomers, or mixtures thereof.

9. The stick composition of claim 2 wherein the polymeric microparticles comprise an allyl methacrylate crosspolymer, an ethylene glycol dimethacrylate/allyl methacrylate copolymer, a lauryl methacrylate/ethylene glycol dimethacrylate copolymer, a methyl methacrylate/glycol dimethacrylate copolymer, and mixtures thereof.

10. The stick composition of claim 2 wherein the polymeric microparticles are selected from the group consisting of a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

11. The stick composition of claim 2 wherein the polymeric microparticles comprise a copolymer of allyl methacrylate and ethylene glycol dimethacrylate, a copolymer of ethylene glycol dimethacrylate and lauryl methacrylate, or a mixture thereof.

12. The stick composition of claim 2 wherein the polymeric microparticles comprise copolymer of ethylene glycol dimethacrylate and lauryl methacrylate.

13. The stick composition of claim 4 wherein the loaded microparticle delivery system comprises about 10% to 90%, by weight, of the active agent.

14. The stick composition of claim 4 wherein the active agent is present in the composition in an amount of about 0.05% to about 10% by weight.

15. The stick composition of claim 1 wherein the microparticle delivery system further comprises a barrier layer.

16. The stick composition of claim 15 wherein the barrier layer is present in an amount of about 1% to about 75%, by weight of the loaded microparticle delivery system.

17. The stick composition of claim 4 wherein the active agent is a tooth-whitening agent.

18. The stick composition of claim 17 wherein the tooth-whitening agent comprises hydrogen peroxide, carbamate peroxide, sodium percarbonate, sodium perborate, potassium peroxydiphosphate, an organic peracid, or mixtures thereof.

19. The stick composition of claim 4 wherein the active agent is an anti-acne agent.

20. The stick composition of claim 19 wherein the anti-acne agent comprises salicylic acid, benzoyl peroxide, resorcinol, sulfur, retinoic acid, or mixtures thereof.

21. The stick composition of claim 4 wherein the active agent is a skin-treatment agent.

22. The stick composition of claim 21 wherein the skin-treatment agent comprises retinol, retinol palmitate, retinol acetate, dihydroxyacetone, petrolatum, arbutin, ascorbic acid and derivatives thereof, tocopherol and derivatives thereof, L-erythrulose, hydroquinone, kojic acid, kojic dipalmitate, dimethicone, or mixtures thereof.

23. The stick composition of claim 1 in the form of a solid or a gel.
